(19) United States
(12) Patent Application Publication
(10) Pub. No.: US 2023/0061669 A1
(43) Pub. Date: Mar. 2, 2023

(54) Hoof treatment comprising Chlorine Dioxide

(71) Applicant: Clear Lakes Products, LLC, Buhl, ID (US)

(72) Inventor: James Holesinksy, Buhl, ID (US)

(21) Appl. No.: 17/811,817

(22) Filed: Jul. 11, 2022

Related U.S. Application Data
(60) Provisional application No. 63/186,654, filed on May 10, 2021.

(51) Int. Cl. A61K 33/20; A61K 9/00

(52) U.S. Cl. CPC A61K 33/20; A61K 9/0014

(57) ABSTRACT

Bacterial infections in dairy cattle are treated by applying a solution of acidified sodium chlorite/chlorine dioxide to the hooves of the animals. Preferably, the solution is applied to the hooves by placing the solution in a foot bath and guiding the animals through the foot bath.

### CROSS-REFERENCE TO RELATED APPLICATIONS

This application claims the benefit of U.S. Provisional Application No. 63/186,654, filed May 10, 2021 the disclosure of which is incorporated by reference.

### FIELD OF THE INVENTION

The presently disclosed technology relates to a method of treating and/or preventing a bacterial infection in the hooves of livestock. More particularly, the present invention is a method of treating hooves by placing a solution utilizing acidified sodium chlorite/chlorine dioxide in a foot bath and guiding the animal's hooves through the foot bath.

### BACKGROUND OF THE INVENTION

Dairy cattle are prone to bacterial hoof infections that can be quite debilitating. A common condition in cattle is “foot rot” or what is medically termed interdigital phlegmon. It is an infection of the soft tissue between the claws of the feet and is caused by two anaerobic bacteria, Fusobacterium necrophorum and Prevotella melaninogenicus (formerly known as Bacteroides melaninogenicus). These bacteria are common in the environment and F. necrophorum is present in the rumen and feces of normal cattle. Once these bacteria invade the skin of the foot, they rapidly cause the condition recognized as foot rot. Injury or damage to the skin between the claws allows this invasion to occur. Common factors that can cause damage of this sort include stubble fields, small rocks and pebbles, and abrasive surfaces. Additionally, high temperatures and excess moisture or humidity causes the skin between the claws to chap and crack allowing these bacteria to invade.

Farmers typically take steps to avoid foot rot. One common practice is to utilize a foot bath for cattle. This is not only an effective preventive measure, it is typically an effective treatment for such bacterial infections.

There are three different types of solutions commonly used in foot baths: Formaldehyde, zinc sulfate and copper sulfate. For treatment, the footbath should be used 1-2 times per week for several weeks. The footbath may also be used routinely after foot trimming and as a preventative. Predominantly, copper sulfate and formaldehyde are the solutions of choice for treating foot rot, particularly with cattle. Zinc sulfate, although somewhat effective, has shown mixed results.

The use of copper in the form of copper sulfate presents environmental concerns. The foot bath solution is typically drained into the manure store and is subsequently applied to the fields. Excess copper can be hazardous to the cattle and sheep. The application of the copper-laden manure onto the fields causes a copper build-up, which can be problematic to the plants' development and, therefore, should be avoided.

### SUMMARY

The purpose of the Summary is to enable the public, and especially the scientists, engineers, and practitioners in the art who are not familiar with patent or legal terms or phraseology, to determine quickly from a cursory inspection, the nature and essence of the technical disclosure of the application. The Summary is neither intended to define the inventive concept(s) of the application, which is measured by the claims, nor is it intended to be limiting as to the scope of the inventive concept(s) in any way.

The present invention is premised on the discovery that bacterial hoof infections of cows can be treated with an aqueous solution of acidified sodium chlorite/chlorine dioxide. More particularly, the present invention involves the application of acidified sodium chlorite/chlorine dioxide, typically using a foot bath, to effectively treat and prevent foot rot and other bacterial infections in cattle. The objects and advantages of the present invention will be further appreciated in light of the following detailed description.

Still other features and advantages of the presently disclosed and claimed inventive concept(s) will become readily apparent to those skilled in this art from the following detailed description describing preferred embodiments of the inventive concept(s), simply by way of illustration of the best mode contemplated by carrying out the inventive concept(s). As will be realized, the inventive concept(s) is capable of modification in various obvious respects all without departing from the inventive concept(s). Accordingly, the drawings and description of the preferred embodiments are to be regarded as illustrative in nature, and not as restrictive in nature

### DETAILED DESCRIPTION OF THE INVENTION

While the presently disclosed inventive concept(s) is susceptible of various modifications and alternative constructions, certain illustrated embodiments thereof have been shown in the drawings and will be described below in detail. It should be understood, however, that there is no intention to limit the inventive concept(s) to the specific form disclosed, but, on the contrary, the presently disclosed and claimed inventive concept(s) is to cover all modifications, alternative constructions, and equivalents falling within the spirit and scope of the inventive concept(s) as defined in the claims.

Bacterial infections in cattle and other livestock are treated with an aqueous solution of acidified sodium chlorite/chlorine dioxide. acidified sodium chlorite/chlorine dioxide can be obtained as an aqueous solution.

The effective concentration of the acidified sodium chlorite/chlorine dioxide solution is preferably 100-10,000 PPM by volume for treating existing infections.

The preferred method to apply the solution of acidified sodium chlorite/chlorine dioxide to the hooves of the cattle is the use of a walk through foot bath. More particularly, the animals are simply directed through the foot bath. Preferably, the foot baths will be about 6 inches deep of the acidified sodium chlorite/chlorine dioxide so the solution should be filled to a depth of at least 4 inches. The foot bath should be 8 to 12 feet long and is preferably preceded by a wash bath containing only water, or water and detergent, to remove manure from the feet prior to passing through the treatment bath. The treatment bath will typically be drained to the manure store.

Preferably, the chemical in the treatment bath is changed after about 200 animals have passed through the bath. The bath should then be thoroughly rinsed and cleaned prior to subsequent use.

The manure store with the drained acidified sodium chlorite/chlorine dioxide solution can then be drained.

While certain preferred embodiments are shown in the figures and described in this disclosure, it is to be distinctly understood that the presently disclosed inventive concept(s) is not limited thereto but may be variously embodied to practice within the scope of the following claims. From the foregoing description, it will be apparent that various changes may be made without departing from the spirit and scope of the disclosure as defined by the following claims.

What is claimed is:

1. A method of treating hooves of livestock to reduce bacterial infections in said hooves comprising:
The step of applying a treatment solution comprising of an aqueous solution of acidified sodium chlorite/chlorine dioxide onto said hooves of said cattle.

2. The method claimed in claim 1 wherein said treatment solution contains about 100-100,000 PPM of acidified sodium chlorite/chlorine dioxide by volume.

3. The method claimed in claim 2 wherein method comprises the step of placing said treatment solution is placed in a foot bath and the animals are directed through the foot bath.

4. The method claimed in claim 3 wherein said solution of sodium chlorite in said foot bath is activated with an acid, wherein said acid comprising at least one of hydrochloric acid, citric acid, phosphoric acid, mineral acids, zinc chloride, acidified copper, formalin, organic acids, acid based surfactants, sulfuric acid, natural and synthetic acids.

5. The method claimed in claim 1 wherein said solution has a pH of less than 7.

6. A method of treating hooves of animals to reduce bacterial infections in hooves of an ungulate comprising applying to the hooves of an ungulate a treatment solution comprising acidified sodium chlorite/chlorine dioxide and water onto said hooves of said animals.

7. A method of treating hooves of cattle to reduce bacterial infections in said hooves comprising:
a step of applying a treatment solution consisting of an aqueous solution of acidified sodium chlorite/chlorine dioxide onto said hooves of said cattle.

8. (canceled)
